(19) United States
(12) Patent Application Publication
(10) Pub. No.: US 2009/0012164 A1
(43) Pub. Date: Jan. 8, 2009

(54) PINOLENIC ACID COMPOSITIONS, PRODUCTS MADE THEREOF, AND METHODS OF MAKING PINOLENIC ACID COMPOSITIONS AND PRODUCTS

(76) Inventor: Erik Kelderman, Almere (NL)

Correspondence Address: ARIZONA CHEMICAL COMPANY;c/o Bo Segers, P.O. Box 550850, Jacksonville, FL, 32255, US

(21) Appl. No.: 12/136,026

(22) Filed: Jun. 9, 2008

Related U.S. Application Data
(60) Provisional application No. 61/130,467, filed on Jun. 9, 2007.

(51) Int. Cl. A61K 31/20; A61P 43/00; A23L 2/00; A23L 1/48

(52) U.S. Cl. 514/558; 426/590; 426/531

(57) ABSTRACT

Compositions of pinolenic acid or derivatives thereof. Method of using TOFA for obtaining pinolenic acid or derivatives thereof. Products, including foods, beverages, personal care, beauty, pharaceutical or other products comprising TOFA derived pinolenic acid or derivatives thereof.

### RELATED APPLICATION DATA

This application claims priority from U.S. Provisional Patent Application No. 61/130,467, filed Jun. 9, 2007, and herein incorporated by reference.

### BACKGROUND OF THE INVENTION

1. Field of the Invention

The present invention relates to fatty acids, to methods of processing fatty acids, to methods of using fatty acids, to products comprising fatty acids, and to methods of making and using such products. The present invention relates to polyunsaturated fatty acids, to methods of processing polyunsaturated fatty acids, to methods of using polyunsaturated fatty acids, to products comprising polyunsaturated fatty acids, and to methods of making and using such products. In another aspect, the present invention relates to linolenic and/or pinolenic acids, to methods of processing linolenic and/or pinolenic acids, to methods of using linolenic and/or pinolenic acids, to products comprising linolenic and/or pinolenic acids, and to methods of making and using such linolenic and/or pinolenic acid products.

2. Description of the Related Art

Pine nuts have been eaten in Europe and Asia since the Paleolithic period. They are frequently added to meat, fish, and vegetable dishes. Pine nut coffee, known as pinon (Spanish for pine nut), is a speciality found in the southwest United States, especially New Mexico; it is typically a dark roast coffee and has a deep, nutty flavor. Pine nuts are also used in chocolates and desserts such as baklava.

Nutritionally, pine nuts contain about 31 grams of protein per 100 grams of nuts, the highest of any nut or seed. They are also a source of dietary fibre.

Pine nuts can be pressed to extract pine nut oil, which is valued both for its mild, nutty flavour and its purported health benefits such as appetite suppression and antioxidant action. Pine nut oil also had economic importance in pre-revolution Russia.

Pinolenic acid is an active ingredient present in all 140 varieties of pine nuts (and their oil) in quantities ranging from 0.1 to more than 20 percent. Chemically, pinolenic acid is a triple-unsaturated fatty acid with 18 carbon atoms having three double bonds in positions 5, 9 and 12. It is the positional isomer of a more widely known gamma-linolenic acid (GLA).

There is interest in pinolenic acid, and ester and other derivatives thereof, as it is known to have a number of beneficial uses.

For example, it is known that pine nut oil (and thus pinolenic acid) can be added to food products, specifically through the use of a food additive concentrate comprising pinolenic acid. The presence of pinolenic acid provides a hypolipemic effect, that is, it lowers the concentration of fats in the blood.

Further the prior art indicates that pinolenic acid derivatives have a number of health benefits. WO 9843513 discloses that nail files can be coated with pinolenic acid and that this inhibits the occurrence of infections upon use of the files. Any patents, applications, publications cited herein, are hereby incorporated by reference.

JP 61238729 discloses that pine oil can be used as anticholesterimic agent.

JP 61058536 discloses the pine oil can provide a very generic activity beneficial for human health.

Sugano in Brit J. of Nutr 72 (1994) 775-783, reports pinolenic acid as effecting ADP-induced platelet aggregation, aortic prostacylic production, and blood pressure, and as providing hypocholesterolaemic effects.

Matsuo discloses pinolenic acid as having an effect on CD-4″-lymphocytes and on CD8+-subsets, in Prostagl, Leukotrienes and Essential fatty Acids 55 (1996) 223-229.

EP 1 088 552, published Apr.19, 2006, and U.S. Pat. No. 6,479,070, issued Nov. 12, 2002 both disclose pinolenic acid as a food composition, a food supplement, or a pharmaceutical composition, providing anti-inflammatory properties.

Most recent interest in pinolenic acid centers around the use of pinolenic acid for the treatment of obesity. Research has shown its potential use in weight loss by curbing the appetite. Specifically, it is believed that pinolenic acid causes the triggering of two hunger suppresants—cholecystokinin and glucagon-like peptides.

EP 1 685 834, published Feb. 8, 2002, discloses that pinolenic acid or a derivative thereof can be used for weight management.

U.S. Pat. No. 6,809,115, issued Oct. 24, 2006, discloses a pinolenic-containing composition for reducing body fat, as well as treating insulin-dependent diabetes, improving insulin sensitivity, reducing hyperglycemia, and reducing hypercholesterolemia. The composition is described as containing at least one chromium complex and a conjugated fatty acid or conjugated fatty alcohol (with pinolenic acid listed as an example).

In Europe, pine nuts come from the Stone Pine (Pinus pinea), which has been cultivated for its nuts for over 6,000 years, and harvested from wild trees for far longer. The Swiss Pine (Pinus cembra) is also used to a very small extent.

In Asia, two pine species are widely harvested. The first is the Korean Pine (Pinus koraiensis) in northeast Asia, which is the most important species in international trade. The second is the Chilgoza Pine (Pinus gerardiana) in the western Himalaya. Four other species, Siberian Pine (Pinus sibirica), Siberian Dwarf Pine (Pinus pumila), Chinese White Pine (Pinus annandii) and Lacebark Pine (Pinus bungeana), are also used to a lesser extent.

In United States and Mexico, the main species are three of the pinyon pines: Colorado Pinyon (Pinus edulis), Single-leaf Pinyon (Pinus monophylla) and Mexican Pinyon (Pinus cembroides). The other eight pinyon species are used to a small extent, as are Gray Pine (Pinus sabineana), Torrey Pine (Pinus torreyana) and Sugar Pine (Pinus lambertiana).

Obviously, the amount of available pine nuts is limited by the number of suitable pine trees. Piney woods have been lost due to conversion to grazing lands, and because of destructive harvesting techniques (such as breaking off whole branches to harvest the cones). The harvesting of trees for timber have led to losses in pine nut production capacity. Thus, the amount of pine nuts available for extraction of the oils is very limited. Therefore, the prices of the oils are already very high. It is very likely, that the positive health effects of pinolenic acid will provide pressure on the supplies of the acid.

A number of patents are directed to the fatty acids, including the following.

U.S. Pat. No. 3,860,569, issued Jan. 14, 1975, to Ward, discloses a process for treating tall oil fatty acids. The process comprises treating tall oil fatty acids with a bromine-iodine catalyst to convert the linoleic acid portion to oleic and other acids. The catalyst is employed in an amount of 0.06 percent to 2.0 percent by weight fatty acids at a temperature between 400.degree.F. and 550.degree.F. for a period of time between 10 minutes and 6 hours. The bromine-iodine catalyst is used at a bromine to iodine ratio of between 1:1 to 5:1 parts by weight. Tall oil fatty acids treated with the bromine-iodine catalyst of this invention give fatty acids almost identical to fatty acids treated with iodine alone. The advantage of this process is that less iodine catalyst is used to accomplish equivalent results as with iodine alone.

U.S. Pat. No. 3,884,046, issued May 20, 1975, to Schmidt, et al., discloses a method of separating components of fatty-acid, fatty-alcohols and fatty-acid-ester mixtures thereof in which an aqueous surfactant solution is intimately mixed with the fatty-acid, fatty-alcohol or fatty-acid-ester mixture to form an emulsion and, in one or more stages, reduced pressure(suction) is applied to evaporate water and reduce the temperature to the crystallization level. Preferably the evaporation of water is carried out in several stages, the aqueous surfactant being supplied all at once or in several stages. The crystallized component is then recovered by solid-liquid separation.

U.S. Pat. No. 3,950,365, issued Apr. 13, 1976, to Singer, et al., discloses a method for the purification of mixtures of fatty acids or fatty acid esters containing polyunsaturated components by heating the mixture in the presence of an organic macroporous, acid ion exchange resin having a specific surface area of at least 35 m.sup.2/ gm and devoid of gel characteristics, and then separating the purified mixture by distillation. The method is especially useful for the production of oleic acid which is relatively free of linoleic acid.

U.S. Pat. No. 3,950,371, issued Apr. 13, 1976, to Jeromin, et al., discloses a method for separating fatty substance mixtures into components of different melting points by the “Rewetting or Hydrophilization Process,” with the heat removal necessary for cooling and crystallizing higher melting fatty substance fractions being obtained essentially by vacuum evaporation of an aqueous, non-surface-active electrolyte solution in direct contact with the fatty substance mixture.

U.S. Pat. No. 3,953,484, issued Apr. 27, 1976, to Sutker, discloses fractionation of liquid or solid mixtures of fatty acids is enhanced by a pretreatment which comprises whipping and maceration of the mixture to form a gas-entrained slurry. Preferably the whipping and maceration are carried out in the presence of air. During the pretreatment normally solid fatty acid mixtures are slurrified and the obtained slurry is filterable to separate liquid and solid fatty acid fractions therefrom.

U.S. Pat. No. 4,048,205, issued Sep. 13, 1977, to Neuzil, et al., discloses a process for separating an ester of a monoethanoid fatty acid from a mixture comprising an ester of a saturated fatty acid and an ester of an unsaturated fatty acid consisting essentially of an ester of a monoethanoid fatty acid which process comprises contacting the mixture at adsorption conditions with an adsorbent comprising a X or a Y zeolite containing selected cations at the exchangeable cationic sites thereby selectively adsorbing the ester of a monoethanoid fatty acid. Preferably the ester of a monoethanoid fatty acid will be recovered from the adsorbent by desorption with a desorbent material.

U.S. Pat. No. 4,049,688, issued Sep. 20, 1977, to Neuzil, et al., discloses a process for separating an ester of an unsaturated fatty acid from a mixture comprising an ester of an unsaturated fatty acid and an ester of a saturated fatty acid which process comprises contacting the mixture at adsorption conditions with an adsorbent comprising a X or a Y zeolite containing one or more selected cations at the exchangeable cationic sites thereby selectively adsorbing the ester of an unsaturated fatty acid. Preferably the ester of an unsaturated fatty acid will be recovered from the adsorbent by desorption with a desorbent material.

U.S. Pat. No. 4,066,677, issued Jan. 3, 1978, to de Rosset, et al., discloses a process for separating an ester of a polyethanoid fatty acid and an ester of a monoethanoid fatty acid from a mixture comprising an ester of a polyethanoid fatty acid, an ester of a monoethanoid fatty acid and an ester of a saturated fatty acid which process comprises contacting the mixture at first adsorption conditions with a first adsorbent comprising a X or a Y zeolite containing a selected cation at the exchangeable cationic sites thereby selectively adsorbing the ester of a polyethanoid fatty acid and thereafter recovering the ester of a polyethanoid fatty acid; removing from the first adsorbent a second mixture comprising an ester of a monoethanoid fatty acid and an ester of a saturated fatty acid; contacting the second mixture at second adsorption conditions with a second adsorbent comprising a X or a Y zeolite containing selected cations at the exchangeable cationic sites thereby selectively adsorbing the ester of a monoethanoid fatty acid and thereafter recovering the ester of a monoethanoid fatty acid. Preferably the polyethanoid fatty acid ester and a monoethanoid fatty acid will be recovered from the first and second adsorbents respectively by desorption with a first and a second desorbent material.

U.S. Pat. No. 4,329,280, issued May 11, 1982, to Cleary, et al., discloses a process for separating an ester of a fatty acid from a mixture comprising an ester of a fatty acid and an ester of a rosin acid, which process comprises contacting the mixture at adsorption conditions with an adsorbent comprising silicalite, thereby selectively adsorbing the ester of a fatty acid. Preferably the ester of a fatty acid will be recovered from the adsorbent by desorption with a desorbent material.

U.S. Pat. No. 4,353,839, issued Oct. 12, 1982, to Cleary, et al., discloses a process for separating a one saturated fatty acid from a mixture of saturated fatty acids, which process comprises contacting the mixture at adsorption conditions with an adsorbent comprising a hydrophobic insoluble crosslinked polystyrene polymer, thereby selectively adsorbing the saturated fatty acid for which the adsorbent is selective. Preferably the adsorbed saturated fatty acid will be recovered from the adsorbent by desorption with a desorbent material.

U.S. Pat. No. 4,443,437, issued Apr. 17, 1984, to Prokosch et al., discloses a topical veterinary composition and method of using same for the treatment of flesh wounds or lacerations or fistulas in animals and to promote the healing thereof. The composition comprises as the active ingredient tall oil either per se or as a topical veterinary ointment comprising the active ingredient in admixture with a suitable carrier and/or antiseptic.

U.S. Pat. No. 4,404,145, issued Sep. 13, 1983, to Cleary, et al., discloses a process for separating a fatty acid from a mixture comprising a fatty acid and a rosin acid, which process comprises contacting the mixture at separation conditions with a molecular sieve comprising silicalite, thereby selectively retaining the fatty acid. The fatty acid may be recovered from the molecular sieve by displacement with a displacement fluid. It is preferred that the displacement fluid comprise a liquid having a minimum polarity index.

U.S. Pat. No. 4,444,986, issued Apr. 24, 1984, to Dessau, discloses an improved hydrocarbon separation process by the selective sorption properties of certain members of a novel class of zeolites is provided. The novel class of zeolites is characterized by a silica to alumina mole ratio greater than 12 and a Constraint Index within the approximate range of greater than about 2 to about 12. The process of this invention involves selective separation of higher molecular weight hydrocarbon compounds in admixture with lower molecular weight hydrocarbon compounds within a homolgous series by contacting the respective mixture with a zeolite having a SiO.sub.2/A1.sub.2 O.sub.3 mole ratio of at least about 12 and a Constraint Index with the approximate range of greater than 2 to about 12, to effect the selective sorption of said higher molecular weight.

U.S. Pat. No. 4,495,106, issued Jan. 22, 1985, to Cleary, et al., discloses a process for separating a fatty acid from a mixture comprising a fatty acid and a rosin acid, which process comprises contacting the mixture at separation conditions with a molecular sieve comprising silicalite in a silica matrix the precursor of the molecular sieve comprising silicalite powder dispersed in colloidal amorphous silica, the precursor having been gelled and then treated in a manner to substantially eliminate hydroxyl groups from the molecular sieve, thereby selectively retaining the fatty acid. The fatty acid is recovered from the molecular sieve by displacement with a displacement material comprising an organic acid.

U.S. Pat. No. 4,511,514, issued Apr. 16, 1985, to Cleary, et al., discloses a process for separating oleic acid from a feed mixture comprising an oleic acid and linoleic acid, which process comprises contacting the mixture at separation conditions with a molecular sieve comprising silicalite, thereby selectively retaining the oleic acid. The oleic acid may be recovered from the molecular sieve by displacement with a displacement fluid. The feed mixture may also contain rosin acids in which case a first molecular sieve comprising silicalite is used to separate the fatty acids from the rosin acids.

U.S. Pat. No. 4,524,030, issued Jun. 18, 1985, to Cleary, et al., discloses a process for separating a fatty acid from a mixture comprising a fatty acid and a rosin acid, which process comprises contacting the mixture at separation conditions with a molecular sieve comprising a crystalline silica, thereby selectively retaining the fatty acid. The fatty acid is recovered from the molecular sieve by displacement with a displacement fluid comprising an ester containing less than six carbon atoms per molecule. Once the rosin acid is removed from the feed mixture, the process is also effective in separating the fatty acids from each other, using the same molecular sieve and displacement fluid.

U.S. Pat. No.4,529,551, issued Jul. 16, 1985, to Cleary, et al., discloses a process for separating oleic acid from a feed mixture comprising an oleic acid and linoleic acid, which process comprises contacting the mixture at separation conditions with a molecular sieve comprising silicalite, thereby selectively retaining the oleic acid. The oleic acid is recovered from the molecular sieve by displacement with a displacement fluid comprising a diluent soluble in the feed mixture and having a polarity index of at least 3.5. Displacement occurs at a temperature from about 120.degree. C. to about 150.degree. C. The feed mixture may also contain rosin acids in which case a first molecular sieve comprising silicalite is used to separate the fatty acids from the rosin acids.

U.S. Pat. No. 4,534,900, issued Aug. 13, 1985, to Cleary, discloses a process for separating a fatty acid from an unsaponifiable compound. A feedstream comprising the acid and unsaponifiable compound is contacted with an aqueous alcohol solvent which is selective for and absorbs the fatty acid. An extract stream comprising the fatty acid may then be recovered. The feedstock is best used in a diluent which is preferably a hydrocarbon.

U.S. Pat. No. 4,578,223, issued Mar. 25, 1986, to Cleary discloses a process for separating a first saturated fatty acid from a second saturated fatty acid contained in a feed mixture comprising the acids, the chain length of the first being at least two carbon atoms greater than that of the second. The process comprises contacting the feed mixture at adsorption conditions comprising a crystalline silica having a silica to alumina mole ratio of at least 12, thereby selectively adsorbing the first saturated fatty acid. The remainder of the feed mixture is then removed from the adsorbent, and the first acid recovered from the adsorbent by desorption at desorption conditions with a desorbent liquid soluble in the feed mixture and having a polarity index of at least 3.5.

U.S. Pat. No. 4,601,856, issued Jul. 22, 1986, to Suzuki, et al., discloses a method of producing a highly purified oleic acid is disclosed, which comprises the steps of: (a) separating and removing the resulting precipitated crystal after the cooling of a solution of oleic acid containing fatty acid mixture and urea in an organic solvent; (b) separating the resulting crystallized crystal after the partial saponification of the organic solvent solution; and (c) subjecting the crystal to an acid decomposition.

U.S. Pat. No. 5,179,219, issued Jan. 12, 1993, to Priegnitz, discloses the separation of free fatty acids from triglycerides is performed by an adsorptive chromatographic process in liquid phase with silica gel as the adsorbent. A ketone, having from 3 to 8 carbon atoms, such as 2-heptanone, an ester or an ether can be selected as the desorbent

U.S. Pat. No. 5,194,640, issued Mar. 16, 1993, to Cosgrove, et al., discloses an oleic acid containing adduct is produced by reacting tall oil fatty acid (which contains pre-conjugated linoleic acid) with a dienophile at a temperature between 180.degree. C. and 300.degree. C. No catalyst or solvent is necessary for the reaction to occur. The adduct is subsequently distilled to yield a high-purity, light color oleic acid.

U.S. Pat. No. 5,225,580, issued Jul. 6, 1993, to Zinnen, discloses a two-stage separation process for separating highly unsaturated triglycerides from an interesterification reaction product by sequential chromatographic separations wherein triglycerides are separated from saturated fatty acids in the first stage and unsaturated fatty acids are recovered in the second stage for recycle to the interesterification reaction zone. In a preferred embodiment using silica gel and silicalite in the two stages, respectively, and heptanone as desorbent in both stages, triglyceride product is removed as the first stage raffinate and the unsaturated fatty acid recycle product stream is removed as the raffinate in the second stage. In another embodiment, using silicalite adsorbent in both stages and 2-heptanone and acetone in the first and second stages, respectively, saturated fatty acids are removed as the extract in the first stage and triglycerides and unsaturated fatty acids are separated as raffinate product and extract product for recycle, respectively.

International Publication No. WO 02/41865, published May 30, 2002, discloses a cosmetic method for treating aged, sensitive, dry, flaky, wrinkled and/or photo-damaged skin through topical application of a composition which comprises pinolenic acid and/or derivatives thereof. The invention also relates to compositions suitable for such cosmetic treatment.

EP 1,685,834, published Aug. 2, 2006, discloses a pinolenic acid or derivative thereof useful for weight management. The pinolenic acid may, for example, be used in the form of a food supplement, a pharmaceutical composition or a food composition.

U.S. Patent Application Publication No. 2006/0257333, published Nov. 16, 2006, discloses skin care products in oil, cream, emulsion, gel, liquid and stick form for dry and scaling skin. The products comprise 1-90% by weight of tall oil fatty acids and 99-10% by weight of various vegetable oils and their fatty acids. Further, the products may contain emulsifiers, thickeners, solvents and powdery flours, depending on the purpose of use of the product on various parts of the skin.

### SUMMARY OF THE INVENTION

The following presents a general summary of some of the many possible embodiments of this disclosure in order to provide a basic understanding of this disclosure. This summary is not an extensive overview of all embodiments of this disclosure. This summary is not intended to identify key or critical elements of the disclosure or to delineate or otherwise limit the scope of the claims. The following summary merely presents some concepts of the disclosure in a general form as a prelude to the more detailed description that follows.

A non-limiting and non-exhaustive list of embodiments of the invention include:
a. A composition comprising pinolenic acid (or ester or other derivative thereof) and in the range of about 0.1 to about 4 wt % C16:0.
b. A composition comprising pinolenic acid (or ester or other derivative thereof) and either none, or optionally in the range of about 0.1 to about 10 wt % rosin.
c. A composition comprising pinolenic acid (or ester or other derivative thereof) and at least 0.1 ppm sulfur.
d. A composition comprising pinolenic acid (or ester or other derivative thereof) and in the range of about 0.1 to about 200 ppm sulfur.
e. Products comprising pinolenic acid (or ester or other derivative thereof) as described herein, wherein products comprise beverages, foods, supplements, pharmaceuticals, personal health products, or beauty products.
f. Methods of making products comprising pinolenic acid (or ester or other derivative thereof) as described herein, wherein products comprise beverages, foods, supplements, pharmaceuticals, personal health products, or beauty products.
g. A food product comprising pinolenic acid (or ester or other derivative thereof) and in the range of about 0.1 to about 4 wt % C16:0.
h. A food product comprising pinolenic acid (or ester or other derivative thereof) and either none, or optionally in the range of about 0.1 to about 10 wt % rosin.
i. A food product comprising pinolenic acid (or ester or other derivative thereof) and at least 0.1 ppm sulfur.
j. A food product comprising pinolenic acid (or ester or other derivative thereof) and in the range of about 0.1 to about 200 ppm sulfur.
k. A beauty product comprising pinolenic acid (or ester or other derivative thereof) and in the range of about 0.1 to about 4 wt % C16:0.
l. A beauty product comprising pinolenic acid (or ester or other derivative thereof) and either none or in the range of about 0.1 to about 10 wt % rosin.
m. A beauty product comprising pinolenic acid (or ester or other derivative thereof) and in the range of about 0.1 to about 200 ppm sulfur.
n. A beauty composition comprising pinolenic acid (or ester or other derivative thereof) and at least 0.1 ppm sulfur.
o. Use of TOFA derived pinolenic acid or derivative thereof in the manufacture of a Product comprising pinolenic acid (or ester or other derivative thereof) as described herein, wherein products comprise beverages, foods, supplements, pharmaceuticals, personal health products, or beauty products.
p. Use of TOFA derived pinolenic acid or derivative thereof in the manufacture of a foodstuff.
q. Use of TOFA derived pinolenic acid or derivative thereof in the manufacture of a composition for weight management by reducing the feeling of hunger and/or increasing satiety.
r. Use of TOFA derived pinolenic acid or derivative thereof, in the manufacture of a composition for the treatment or prevention of inflammation wherein the composition is a food composition or a food supplement or a pharmaceutical composition with anti-inflammatory properties.
s. A method comprising contacting TOFA and an alcohol to produce a composition comprising pinolenic acid (or ester or other derivative thereof) and in the range of about 0.1 to about 4 wt % C16:0.
t. A method comprising contacting TOFA and an alcohol to produce a composition comprising pinolenic acid (or ester or other derivative thereof) and either none or in the range of about 0.1 to about 10 wt % rosin.
u. A method comprising contacting TOFA and an alcohol to produce a composition comprising pinolenic acid (or ester or other derivative thereof) and at least 0.1 ppm sulfur.
v. A method comprising contacting TOFA and an alcohol to produce a composition comprising pinolenic acid (or ester or other derivative thereof) and in the range of about 0.1 to about 200 ppm sulfur.
w. Use of TOFA derived pinolenic acid or derivative thereof in the manufacture of a personal care or beauty product.
x. Any compositions, methods, products as disclosed herein.

### BRIEF DESCRIPTION OF THE DRAWINGS

The following drawings illustrate some of the many possible embodiments of this disclosure in order to provide a basic understanding of this disclosure. These drawings do not provide an extensive overview of all embodiments of this disclosure. These drawings are not intended to identify key or critical elements of the disclosure or to delineate or otherwise limit the scope of the claims. The following drawings merely present some concepts of the disclosure in a general form. Thus, for a detailed understanding of this disclosure, reference should be made to the following detailed description, taken in conjunction with the accompanying drawings, in which like elements have been given like numerals.

FIG. 1 is a plot showing Esterification of Sylfat 2 with glycerol at different temperatures. FIG. 2 is shows the GC-chromatogram of fatty acid methyl esters of Pinnothin and Rest 2 after saponification.

### DETAILED DESCRIPTION OF THE INVENTION

While various embodiments have been shown and described, various modifications and substitutions may be made thereto without departing from the spirit and scope of the invention. Accordingly, it is to be understood that the present invention has been described by way of illustrations and not limitation.

The present invention provides a process which utilizes tall oil fatty acid (“TOFA”) as a novel source of poly-unsaturated fatty acids which can be used in products for human use or consumption, non-limiting examples of which include, as a food supplement, a pharmaceutical composition, as part of a food composition with health benefits, or as part of personal health or beauty products.

Thus, the present invention provides an alternative to pine nuts for obtaining pinolenic acid and other beneficial poly-unsaturated fatty acids by using TOFA as a source of these beneficial acids. As a source of beneficial poly-unsaturated fatty acids, TOFA has at least two major advantages over pine nut oil. First, TOFA is already commonly commercially produced in a large optimized process yielding a fatty acid mixture, which coincidently is very similar to the fatty acid mixture present in pine nut oil. Second, since the TOFA mixture is already in the free acid form, it is ideally suited to increase the amount of pinolenic acid and other poly-unsaturated fatty acids by any number of methods, a non-limiting example of which includes urea ethanol extraction method. This free acid form of TOFA has this advantage over (pine nut) oils, that are always in the triglyceride form, which first need to be split to the free acid and free glycerol before they can be enriched in poly-unsaturated fatty acids.

In some embodiments, the mixture of poly-unsaturated fatty acids may be used in the form of the free acid or as glyceride or as an alkyl ester. The poly-unsaturated concentrates may be made by a process or processes, wherein the impurities are removed by distillation, WFE, filtration, esterification, hydrolysis, trans-esterification, crystallization or other suitable methods needed to refine the product to the required level to qualify for food supplement, a pharmaceutical composition, as part of a food composition, or as part of a heath or beauty product.

Methods of the present invention, include methods comprising any method steps as disclosed herein, any combination the method steps as disclosed herein, any order of the method steps as disclosed herein, and may also include all of the method steps as disclosed herein. In the method of the present invention, any suitable TOFA may be utilized. Preferable TOFA's include those with high levels of poly-unsaturated fatty acids. Even more preferable TOFA's are those with high levels of pinolenic acid, especially, to obtain the health benefits attributed to pinolenic acid. Even more preferable TOFA's are those that originate from crude tall oil (“CTO”) from the pulping process of pine trees grown in cold climates. The cold climate stimulates the pine trees to make a large amount of poly-unsaturated fatty acids, including pinolenic acid.

In the practice of the method of the present invention, the selected TOFA may optionally be further enriched in poly-unsaturated fatty acids including pinolenic acid if desired. Certainly, any suitable method may be utilized to enrich the TOFA. As a non-limiting example, a suitable method is urea/ethanol extraction.

In the practice of the present invention, the TOFA is esterified with an alcohol. The different reactivity between fatty acids and rosin acids results in selective esterification of fatty acids only if the conditions are chosen correctly. This is desirable to leave the rosin acids unreacted and provide the possibility for selective removal of the rosin acids. The reaction temperature is chosen to favor reaction of fatty acids over the rosin acids. Ideally, of course, fatty acids will react and none of the rosin acids will react. While it is preferable that none of the rosin acid react, embodiments of this invention anticipate that there may be some amount of rosin acids which will react. Additionally, the reaction temperature should be low enough to avoid isomerisation and dimerisation of the fatty acid chains. As a non-limiting example, a suitable range for a reaction temperature has the low end of the range starting at about 120 C, 130 C, 140 C, 150 C, 160 C, 170 C or 180 C, and has the high end of the range ending at about 180 C, 190 C, 200 C, 210 C, 220 C, or 230 C. Non-limiting examples of suitable ranges include 160 C-200 C, and 170 C-200 C. Certainly, depending upon the reactants and other conditions, reaction temperatures about or below those listed may in some circumstances be utilized.

Any suitable alcohol may be utilized in the practice of the present invention. As non-limiting examples, the alcohol utilized may be a mono-alcohol like ethanol, butanol or 2-ethanol, or a di-alcohol like mono-ethyl en eglycol (MEG), di-ethyleneglycol (DEG) or 1,4-butanediol or many others. As further non-limiting examples, the alcohol may be a tri-alcohol like TMP or glycerol, or a poly-alcohol like penta-erythritol, di-penta etc. In actually testing, the reaction was conducted with mono-ethyleneglycol (MEG) from 170-190 degrees Celsius. Use of MEG-TOFA may require WFE to purify, a step to hydrolyze the free fatty acid and free MEG, and then a step to split the MEG from the fatty acid.

Other non-limiting examples of suitable alcohols include 2-ethyl-hexanol, butanol, capryl alcohol, dipentaerythritol, glycerol, isononanol, mono ethylene glycol, methanol, neopentyl glycol, pentaerythritol, peg 200, 400, 600 (polyethyleneglycol with Mw of 200, 400 or 600), propylene glycol, polypropylene glycol 2000, trimethylol propane, isopropanol, ethanol.

In some embodiments, a catalyst is not utilized. In other embodiment, the reaction may be conducted in the presence of one or more catalysts. Non-limiting examples of suitable catalysts include magnesium acetate, dibutyltin oxide, tetra isopropyl titanate, Methyl sulphonic acid, titanium silicate.

Some catalysts are heterogeneous and can be removed later such as solid Ti silicate. Some homogenous catalyst may not be a problem to use from food or cosmetics point of view to be left in the final product. Or they can be removed by distillation or filtration or may be self destructive and so on.

The temperature range for the reaction of an alcohol with TOFA can be as low as 80 C with the right use of catalyst and other process conditions, and possibly even lower.

When the reaction mixture is heated, depending on the alcohols chosen, either the esters will evaporate first, or the free fatty acids and free rosin acids will evaporate first. In case of the mono-alcohols the fatty esters will most likely be the most volatile. With the multivalent-alcohols on the other hand the free fatty acids and rosin acids will be the most volatile.

The reaction may be carried out under any suitable stoichiometry. The reaction may be carried out at idea stoichiometry, excess alcohol, or excess acid. Preferably, the reaction is carried out with an excess of acid over alcohol, that is, the amount of alcohol added is less than needed to react all the fatty acids and rosin acids. By using excess acid over alcohol, the products obtained are TOFA ester and free unreacted fatty acids and free rosin acids.

When the reaction is completed, the more volatile components in the mixture will be stripped off by distillation or WFE. If a multivalent alcohol is used the volatile components will be the unreacted free fatty acids and free rosin acids. At the same time also other volatile ingredients are stripped off, like neutrals and bad odor products, which will all the TOFA ester to be suitable for human uses. The first part, selecting the suitable grade of TOFA and also the final part of the invention, describing the synthesis and purification are new as far as we know.

Sulfur is sometimes present in the starting TOFA, and certain amounts of sulfur may be present in after the reaction. In some embodiments, the intermediate and/or final product may comprise sulfur, which may be present in any amount. As a non-limiting example, the intermediate and/or final products generally may comprise in the range of about 0.1 to about 200 ppm of sulfur, preferably may comprise in the range of about 0.1 to about 50 ppm sulfur, more preferably may comprise in the range of about 0.1 to about 25 ppm sulfur, and even more preferably may comprise in the range of about 0.1 to about 12 ppm sulfur.

The intermediate and final products may have lower amounts of C16:0 and/or C18:0 saturates as compared to products from pine nuts. As a non-limiting example, the intermediate and/or final products generally may have less than 4 wt % C16:0, preferably may have less than 2 wt %, more preferably may have less than 1 wt %, even more preferably may have less than 0.5 wt %, and still more preferably may have less than 0.3 wt % C16:0. As another non-limiting example, the intermediate and/or final products generally may have less than 2 wt % C16:0, preferably may have less than 1 wt %, more preferably may have less than 0.5 wt %, and even more preferably may have less than 0.1 wt % C16:0. As a non-limiting example, the intermediate and final products may have a ratio of Pinolenic acid (or derivatives thereof):C16:0 greater than about 4:1, 5:1, 6:1, 7:1, 8:1, 10:1, 15:1, 20:1, or 25:1.

The starting TOFA may also comprise some amount of rosin, which may end up in the intermediate and final products. As a non-limiting example, the intermediate and/or final products generally may have no rosin, or may optionally comprise in the range from about 0.001, 0.01 or 0.1 wt % rosin up to about 5 wt %, 10 wt % or 15 wt % rosin. Non-limiting examples of suitable ranges include from about 0.1 to about 15 wt % rosin, from about 0.1 to about 10 wt % rosin, and from about 0.1 to about 5% wt % rosin, from about 0.01 to about 15 wt % rosin, from about 0.001 to about 10 wt % rosin,

The method of the present invention may also include trans-esterification of the glycerol ester. This may be carried out by the addition of glycerol and optionally the removal of the liberated alcohol. The final product may be a mono-, di- or triglyceride ester or a mixture thereof For food purposes, it may be desirable that the product be a triglyceride. For obesity control, it may be desirable that the product be a di-glyceride, which recent research shows digests slower and thus has benefits for obesity control. If the purified free fatty acids are desired then instead of trans-esterification with glycerol, hydrolysis with water is performed.

Products of the present invention include products for human use or consumption containing TOFA-derived poly-unsaturated fatty acids, non-limiting examples of such products include, beverages, foods, supplements, pharmaceuticals, personal health products, or beauty products. Certainly, skin treatment products can be in the form of pharmaceuticals, personal health products, or beauty products (i.e., cosmetics). It is believed that the TOFA-derived fatty acids may be incorporated into such a product at any point in its manufacture.

As used herein, “foods” are to be taken to refer broadly to meat, poultry, vegetables, fruits, grains, or dairy products, and to any product derived from or containing any of the foregoing.

“Meat” as used herein is to be taken to refer broadly to food products derived from livestock or game animals, including the following nonlimiting examples of beef, pork, veal, lamb, mutton, rabbit, venison, boar, and the like. “Poultry” as used herein is to be taken to refer broadly to food products derived from birds, including the following nonlimiting examples of chicken, turkey, pheasant, duck, quail and even the more exotic emu and ostrich. As used herein, “beverage” is to be understood to be any type of drink, nonlimiting examples of which include water, teas, juices, coffees, carbonated drinks, drinks from water and powders or syrups, wines, or beers.

### EXAMPLES

### Example 1

### Glycerol Ester Synthesis of TOFA Containing Pinolenic Acid as Hunger Suppressant

The objective of this example was to investigate the possibility to make a TOFA tri-glyceride, which is also purified for potential use in food applications. Ingredients to be removed are rosin acids and malodor compounds including volatile sulphur species. While not required for embodiments, in this example, the final composition will preferably resemble Korean pine nut oil, specifically for the tri-glyceride and the fatty acid composition. The main interesting fatty acid in Korean pine nut oil and in TOFA is pinolenic acid, which has been recognized to induce satiety after consumption. This can be called a hunger suppressant effect. Pinolenic acid is mainly present in TOFA of trees form polar regions such as Scandinavian Sylfat 2.

The experimental procedures were as follows.

1. Ensure that reactor is clean & dry.

2. Prepare reactor system for distillation to remove water of reaction.

3. Charge the reactor with the TOFA & Glycerol. Record weights

4. Start agitator and nitrogen sparge, preferably sub-surface, blanketing is acceptable.

5. Set the reaction/pot temperature to 180° C. This temperature should be below the reaction temperature of glycerol and rosin acids!

6. Record time to reach reaction temperature.

7. Once reaction temperature is reached measure acid value.

8. Continue monitoring for AV every 2 hours until AV<30 and AV=16 reached. At AV=30 the excess of TOFA is 0.6 molar. At AV=16 the excess of TOFA is 0.3 molar. If AV>16 then not all glycerol has reacted to triglyceride. Some mono- and di-glyceride will also be present. The mono- and di-glyceride can be easier removed by WFE than the triglyceride.

Continue reaction until AV=16 if possible. AV<16 are not expected.

9. Allow Crude ester to cool and discharge from reactor.

10. Check analysis against Crude ester specification (acid value). If within limits proceed to wiped film evaporator (WFE) stage or steam sparge.

11. The removal of excess fatty acids and rosin acids and neutrals requires the use of a WFE or steam sparge. The intent of this process is to remove all impurities. The aim is to make pure triglycerides of only fatty acids.

12. Possibly suitable WFE conditions based on 4inch WFE in Sandame are <1 Mbar vacuum, Feed rates 3-13 Kg/hr, condenser temperature 30 C, Skin temperature 209-214 C (these temperatures give high levels of mono-glyceride in the distillate, lower temperatures 180-200 C may already give adequate separation).

13. WFE conditions will need to be optimised for each WFE used.

14. Sample residue and analyse for final product specification items.

Synthesis of TOFA glycerol esters from Sylfat 2 at 170° C. with 10% excess acid over glycerol yields mainly tri- and di-glycerides of TOFA. Under these conditions almost no polymeric materials and no rosin esters are formed. The fatty acid composition also remains unchanged during the esterification. Subsequent separation of the tri- and di-glycerides from the non-reacted fatty acids and free rosin acids by WFE at 200° C. yields a product with no rosin acid and no malodor products. Also the volatile sulphur is removed by WFE yielding a sulphur level of 20 ppm from 50 ppm in the starting material Sylfat 2. The product smells very similar to sunflower oil. Also the WFE conditions do not alter the fatty acid composition. In conclusion, it can be stated that a TOFA glycerol ester product can be produced synthetically which after purification can yield a product which is similar to the Korean pine nut oil.

A TOFA glycerol ester was produced at 220° C. top temperature in about 13 hours. It was expected that fatty acids will react faster with glycerol than rosin acids. Therefore, an excess of 10% TOFA was used. This would allow the fatty acids to react but not the rosin acids.

STOICHIOMETRY OF THE REACTION

| TOFA + | GLYCEROL = | TRI-GLYCERIDE + | WATER + | EXCESS TOFA |
|---|---|---|---|---|
| 3.3 MOLE | 1.0 MOLE | 1 MOLE | 3 MOLE | 0.3 MOLE |

Subsequent purification of the tri-glyceride mixture (=Feed) was done by a 4 inch WFE. WFE conditions are <1 mbar vacuum, Feed rates 3-13 Kg/hr, condenser temperature 30° C., Skin temperature 220° C. in Test 1 and 200° C. in Test 2, respectively.

The more volatile products in the reaction mixture, like the excess free fatty acids and rosin acids, are stripped off by WFE and end up as the distillate (Dest). The non-volatile products like di- and tri-glycerides are collected as Rest. Depending on the pressure, flow rate and temperature different ratios of Dest versus Rest can be produced. In this example two different conditions were tried at 220° C. and 200° C., respectively for Test 1 and Test 2.

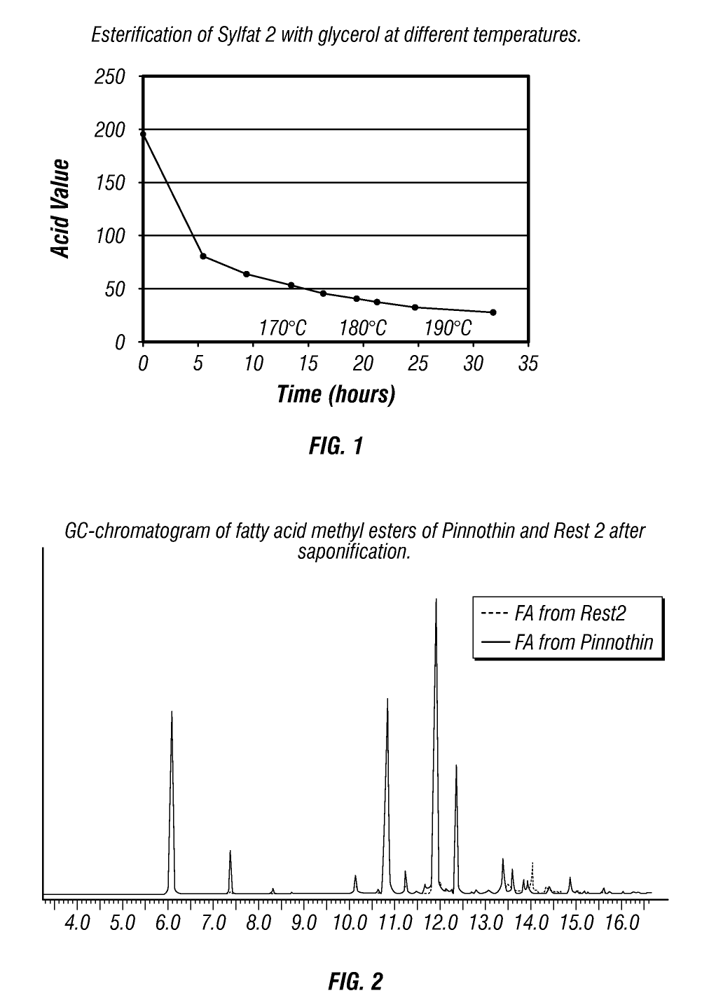

Analysis of the products produced showed that the WFE purification works well. However, the assumption that rosin acids do not react at 220 C proved to be wrong. So depending upon the desired end products and starting mixture, that temperature may or may not be suitable. Additionally, further analysis of the products by GPC showed the presence of molecules with a larger molecular weight than the tri-glyceride. While that may be suitable for some uses, for other uses, that may not be desired. As a result a new synthesis of TOFA tri-glyceride was conducted at lower temperatures in the range from 170-190° C., but using the same stoichiometry of the reaction. For the reaction sequence for the 170-190° C. range, results are provided in FIG. 1. For the first 16 hours the reaction was performed at 170° C. From 16 to 25 hours the reaction was performed at 180° C. After 25 hours the reaction was done at 190° C. The data given in the tables below for 170-190° C. are for the end of the time interval for each temperature.

In this example, the main goal was the removal of free rosin acid and other malodor products from the TOFA tri-glyceride produced. The synthesis at 220° C. and subsequent purification by WFE gave the free rosin acid content as presented in table 1 below. The table nicely shows the removal of all free rosin acids from the Rest fractions under both WFE conditions at 200 and 220° C. Also the free rosin acid content in the distilled fractions (Dest 1 and Dest 2) is high as expected. However, the rosin content of the Feed fraction prior to WFE, produced at 220° C. reaction temperature is surprisingly low at only 0.8%. Subsequent synthesis at lower temperatures from 170-190° C. shows a higher concentration of free rosin acids. On the other hand, synthesis at even higher temperatures of 230° C. shows lower free rosin present. The results presented in Table 1 seem to indicate that (at least for these starting materials) rosin reacts with glycerol to form an ester above 170° C.

TABLE 1
Rosin acid content in some TOFA and tri-glyceride
process streams measured by GPC.

| Sample | Rosin % |
|---|---|
| Sylfat 2 | 2.9 |
| TOFA glycerol | 0.8 |
| 220° C. (=Feed) | |
| Rest 1 (WFE 220° C.) | 0 |
| Rest 2 (WFE 200° C.) | 0 |
| Dest 1 (WFE 200° C.) | 10.0 |
| Dest 2 (WFE 220° C.) | 11.2 |
| TOFA glycerol | 2.8 |
| 170° C. | |
| TOFA glycerol | 2.5 |
| 180° C. | |
| TOFA glycerol | 2.2 |
| 190° C. | |
| TOFA glycerol | 0.8 |
| 220° C. (=Feed) | |
| TOFA glycerol | 0.5 |
| 230° C. (AO-367-105) | |

Besides the rosin content also the sulphur content of the product could be important because of smell and possibly other negative impacts. In Table 2 is shown the overall sulphur content of the products produced, before and after WFE.

TABLE 2
Sulfur contents in TOFA and TOFA
tri-glycerides before and after WFE.

| Sample | Sulfur (ppm) |
|---|---|
| Sylfat 2 | 49 |
| TOFA glycerol | 44 |
| 220° C. (=Feed) | |
| Rest 1 (WFE 220° C.) | 21 |
| Rest 2 (WFE 200° C.) | 20 |
| Dest 1 (WFE 200° C.) | 211 |
| Dest 2 (WFE 220° C.) | 236 |

Table 2 clearly shows the volatile sulphur is stripped off and is collected in the distillate fractions. The non-volatile sulphur remains in the Rest fractions. The amount of 20 ppm of non-volatile sulphur is in line with earlier research.

The Rest fractions are almost odorless and smell similar to sunflower oil. Most probably besides the malodor sulphur products also other low molecular weight malodor products are stripped off by WFE.

Besides the removal of the rosin and malodor products also, in some instances (but not all), the composition of the tri-glycerides should resemble the composition of vegetable oils. In Table 3 the compositions of some vegetable oils, Pinnothin and the TOFA glycerol ester fractions are given. The amounts of free fatty acid and mono-, di-, and tri-glyceride are shown. Also the amount of high molecular weight material is shown. The high molecular weight fractions are often a number of peaks. While not wishing to be limited by theory, it is assumed that these are products produced by either (thermal) dimerisation of the fatty acid chains or by ether formation of the glycerol.

The high molecular weight fractions are also visible in the vegetable oils and Pinnothin product on similar positions in the GPC spectra.

TABLE 3
GPC composition of vegetable oils
and TOFA based glycerol esters.

| | Composition (% area/area) | | | | |
|---|---|---|---|---|---|
| | Mono- | | | | |
| | glyceride | | Di- | Tri- | High MW |
| Sample | (free acid) | | glyceride | glyceride | material |
| Rapeseed oil | 2.7 | | 4.0 | 87.5 | 0.7 |
| Olive oil | 1.0 | | 2.4 | 94.8 | 0.3 |
| Pinnothin | 0.9 | (0.9) | 3.0 | 93.9 | 1.2 |
| TOFA glycerol | 0.6 | (6.4) | 13.8 | 69.3 | 5.1 |
| 220° C. (Feed) | | | | | |
| Rest 1 (WFE 220° C.) | 0.2 | (0.5) | 14.4 | 77.0 | 6.0 |
| Rest 2 (WFE 200° C.) | 0.1 | (0.3) | 14.8 | 78.3 | 6.3 |
| Dest 1 (WFE 200° C.) | 2.0 | (64) | 0.8 | 1.9 | 0 |
| Dest 2 (WFE 220° C.) | 3.4 | (61) | 1.0 | 2.9 | 0 |
| TOFA glycerol | 1.7 | (20) | 24 | 45 | 1.2 |
| 170° C. | | | | | |
| TOFA glycerol | 0.5 | (14.8) | 14.6 | 61.9 | 1.5 |
| 180° C. | | | | | |
| TOFA glycerol | 0.3 | (12.7) | 10.4 | 69.3 | 2.1 |
| 190° C. | | | | | |

| | | | | |
|---|---|---|---|---|
| TOFA glycerol | 2.3 | 6.3 | 76.7 | 12.2 |
| 230° C. (AO-367-105) | | | | |

In table 3 it can be seen that even vegetable oils and Pinnothin do not only consist of tri-glycerides. They also contain mono-, and di-glycerides and even some free fatty acids. The Feed produced at 220° C. has some free acid and mono-glyceride, but this is almost removed in Rest 1 and Rest 2. The free acid and mono-glyceride are collected in the Dest 1 and Dest 2 fractions.

The Rest fractions on the other hand are more concentrated in tri-glyceride content in comparison with the feed.

The high molecular weight fractions in the Feed produced at 220° C. and in the Rest fractions are relatively high at 5.1-6.3%, compared with Pinnothin at 1.2%. This is not desired. When the reactions are done at 170° C. the high molecular weight fraction is the same as for Pinnothin. At higher reaction temperatures the high molecular weight fraction increases rapidly as can be seen in the table.

Combining the information from table 1 to 3 leads to the conclusion that the WFE is suitable for some applications and uses. The synthesis temperature of 220° C. may lead to undesireable products. This high temperature yields 6% polymer and a lot of rosin reacted to glycerol. In order to determine how much rosin has reacted to glycerol Rest 2 was saponified and analyzed. GC analyses shows 1.2% rosin in Rest 2, compared with 2.1% in the starting material Sylfat2. The rosin peaks can be found at retention time 15.6 and higher in the GC chromatogram shown in FIG. 2. Combining these results indicates that at 220° C., about 60-70% of rosin does react with glycerol. Please note that percentages of rosin in GPC and GC do not fully correlate.

For some applications, removal of the undesired products is very important, but the composition of the fatty acids should also not change significantly during the process of esterification and WFE. The composition of the fatty acids of Pinnothin and of the glycerol ester purified by WFE (Rest 2) was compared with that of Sylfat2 by GC. In order to do this the Pinnothin and the ester purified by WFE were first saponified with 2N KOH and subsequently methylated to the methyl esters (FIG. 2). It is known that as a result of saponification and the method used for analysis that some 18:2 and 18:3 products isomerise. Therefore, also a blank of Sylfat 2 was saponified for comparison (Table 4). The isomerisation of 18:2 and 18:3 results in lowering of the Linoleic and Pinolenic peaks. Comparison of the fatty acid composition of saponified Sylfat 2 with the product purified with WFE (Rest 2) in table 4 shows no major differences, with the exception of the rosin content which decreased to 1.2% as mentioned earlier. This shows that the esterification with glycerol and WFE process do probably not significantly change the composition of the fatty acids. Comparison of the Sylfat 2 with Pinnothin at retention time 12.4 shows 8% versus 13% of Pinolenic acid, respectively with the methods of sample preparation and separation according to AQCM022.

TABLE 4
Identification of the major peaks of fatty acid methyl
esters of Sylfat 2 without saponification and Sylfat
2, Rest 2 and Pinnothin with saponification.

| | | Sylfat 2 | Rest 2 | Pinnothin |
|---|---|---|---|---|
| | Sylfat 2 | Saponified | Saponified | Saponified |
| C16:0 Palmitic | 0.3 | 0.3 | 0.3 | 4.1 |
| C16:1 | 0.6 | 0.4 | 0.6 | 0.07 |
| Palmitoleic | | | | |
| C18:0 Stearic | 0.9 | 0.9 | 1.0 | 2.1 |
| C18:1 9-trans | 0.6 | 0.6 | 0.7 | 0.1 |
| Elaidic | | | | |
| C18:1 9-cis Oleic | 26.6 | 25.9 | 27.1 | 24.2 |
| C18:1 isomer | 0.8 | 0.8 | 0.8 | 2.3 |
| C18:2 9,12 | 39.2 | 36.2 | 36.9 | 41.0 |
| Linoleic | | | | |
| C18:3-5,9,12 | 8.0 | 7.0 | 7.2 | 13.2 |
| Pinolenic | | | | |
| C18:2 isomers | 3.8 | 10.0 | 10.0 | 7.0 |
| C20:3-8,11,14 | 1.8 | 1.6 | 1.7 | 1.2 |
| Rosins | 2.1 | 1.9 | 1.2 | 0 |

Finally, in order to produce the TOFA tri-glyceride with 10% excess of fatty acid it will be important to determine how long the reaction will take at different temperatures. At 10% excess of acid the final acid value, the time when all glycerol has reacted with TOFA, is calculated to be about 30 hours. In FIG. 1 is plotted the acid value versus time. For the first 16 hours the reaction was performed at 170° C. From 16 to 25 hours the reaction was performed at 180° C. After 25 hours the reaction was done at 190° C.

From the above, (at least for these reaction conditions) it is clear that the reaction temperature should not be above 170° C. to avoid rosin reacting to glycerol and to avoid formation of large amounts of high molecular weight species (certainly, for some uses higher temperatures may be used). After 16 hours at 170° C. the acid value seems to level off. In table 3 it can be seen that after 16 hours at 170° C. the tri-glyceride content is 45% and the free acid content is still 20%. Knowing that 10% TOFA excess was used means 90% of the reaction is finished after 16 hours. There does not seem to be the need to conduct the reaction longer, since subsequent stripping of the 20% free acid and mono-glyceride by WFE will probably yield the desired TOFA product containing large amounts of di- and mainly tri-glyceride product.

What is claimed is:

1. A composition comprising a pinolenic component comprising at least one selected from pinolenic acid, pinolenic ester, and derivatives of said acid or ester, and optionally in the range of about 0.1 to about 10 wt % rosin.

2. The composition of claim 2, further comprising in the range of about 0.1 to about 4 wt % C16:0.

3. The composition of claim 2, further comprising at least 0.1 ppm sulfur.

4. The composition of claim 3, comprising in the range of about 0.1 to about 200 ppm sulfur.

5. The composition of claim 1, wherein the pinolenic component is derived from TOFA.

6. A product comprising a pinolenic component comprising at least one selected from pinolenic acid, pinolenic ester, and derivatives of said acid or ester, and optionally in the range of about 0.1 to about 10 wt % rosin, wherein the product is in the form of a beverage, foods, supplement, pharmaceutical, personal health product, or beauty products.

7. The product of claim 6, further comprising in the range of about 0.1 to about 4 wt % C16:0.

8. The product of claim 6, further comprising at least 0.1 ppm sulfur.

9. The product of claim 7, comprising in the range of about 0.1 to about 200 ppm sulfur.

10. The product of claim 6, wherein the pinolenic component is derived from TOFA.

11. A method comprising contacting TOFA and an alcohol to produce a composition comprising a pinolenic component comprising at least one selected from pinolenic acid, pinolenic ester, and derivatives of said acid or ester, and optionally in the range of about 0.1 to about 10 wt % rosin.

12. The method of claim 11, wherein the composition further comprises in the range of about 0.1 to about 4 wt % C16:0.

13. The method of claim 11, wherein the composition further comprises at least 0.1 ppm sulfur.

14. The method of claim 13, wherein the composition comprises in the range of about 0.1 to about 200 ppm sulfur.
